(19) United States
(12) Patent Application Publication
(10) Pub. No.: US 2001/0041933 A1
(43) Pub. Date: Nov. 15, 2001

(54) ANNULOPLASTY RING THAT IS RIGID ON IMPLANTATION, BUT BECOMES FLEXIBLE THEREAFTER

(76) Inventor: RANDALL J. THOMA, AUSTIN, TX (US)

Correspondence Address: PHILIP S LYREN PATENT COUNSEL, SULZER MEDICA USA INC, 3 EAST GREENWAY PLAZA, SUITE 1600, HOUSTON, TX, 77046

(21) Appl. No.: 09/316,416

(22) Filed: May 21, 1999

(51) Int. Cl. A61F 2/24

(52) U.S. Cl. 623/2.36

(57) ABSTRACT

The invention, in a first aspect, is an annuloplasty ring comprising a relatively rigid hydrogel insert and a sheath enclosing the hydrogel insert, the sheath being constructed of a biomedical material. The hydrogel insert, prior to implantation, has a water content sufficiently low to provide a desired degree of initial rigidity within the annuloplasty ring. Following surgical implantation of the annuloplasty ring, the water content of the hydrogel insert increases as it absorbs water from surrounding blood and other physiological fluids, thereby increasing the flexibility of the annuloplasty ring. In a second aspect, the invention is a method of manufacturing an annuloplasty ring. The method comprises providing a relatively rigid hydrogel insert and enclosing the hydrogel insert in a sheath constructed from a biomedical material.

### BACKGROUND OF THE INVENTION

1. Field of the Invention

This invention pertains generally to annuloplasty and, more particularly, to an annuloplasty ring.

2. Description of the Related Art

Human heart valves are sometimes damaged by disease or by aging. Such damage may cause problems with the proper function of the leaflets and/or the sub-valvular apparatus attached to the leaflets. Often, degenerative disease causes the valve annulus to enlarge to the point where the leaflets attached to it cannot fully close. This incomplete closure, a condition called valve incompetence, frequently requires surgical correction either by valve repair procedures or by valve replacement. In the former, also called valvular annuloplasty, various types of ring-shaped devices or bands fashioned from biocompatible cloth-like materials are sewn to the distended annulus. By properly sizing and implanting the annuloplasty ring or band, the surgeon can restore the valve annulus to its normal, undilated, circumference.

Annuloplasty rings are typically of two types, either flexible or stiff and comparatively rigid. An example of the former is the Duran Ring or the Cosgrove Band, while an example of the latter is the Carpentier Ring. The Carpentier Ring consists of an open wire element completely covered with cloth. The wire is somewhat stiff yet resiliently deformable and is not intended to be removable from the cloth covering. Carpentier Rings, because of their rigidity, lie flat and maintain their somewhat oval shape during implantation. The flexible Duran Ring and Cosgrove Band consist of a soft core of silicone rubber impregnated with a radiopaque salt, e.g. barium sulfate, completely enclosed by a sheath of biocompatible cloth. The Cosgrove Band mounted on a rigid support is disclosed in U.S. Pat. No. 5,041,130, entitled “Flexible Annuloplasty Ring and Holder,” issued Aug. 21, 1991, to Baxter International, Inc. as the assignee of the inventors Cosgrove et al. The rigid support is subsequently removed after the implanting sutures are tied off.

However, both rigid and flexible annuloplasty rings suffer drawbacks. For instance, although the Carpentier Ring's rigid oval shape is claimed to enhance the competence of the repaired valve, the rigidity also impedes the beneficial flexing movements of the native annulus during the cardiac cycle. Further, because of its flexibility, the Duran Ring is supported during implantation by a holder which is subsequently removed before tying off the implanting sutures, as shown in U.S. Pat. No. 5,011,481, entitled “Holder for Annuloplasty Ring,” issued Apr. 30, 1991, to Medtronic, Inc. as the assignee of the inventors Myers et al. One problem with this approach is that the holder does not completely restrain the entire circumference of the ring and does not prevent the flexible ring from bunching or forming pleats as the implanting sutures are tied off. Still further, neither the Duran Ring or the Cosgrove Band can be tested for competence in the ideal systolic shape as can the rigid Carpentier Ring.

To overcome the deficiencies of flexible and rigid ring structures, an annuloplasty ring would desirably be stiff during handling and implantation, but then become flexible immediately after implant. Also, a surgeon may sometimes desire that the ring could be left rigid for testing the adequacy of his repair, such as by injecting fluid through the opening between the leaflets. This type of testing best conducted with the ring's annulus in its normally oval shape during systole.

Hybrids of the foregoing types of rings have also been proposed to address this problem. For example the Sculptor ring in which the anterior segment (which corresponds to the intertrigone area) is rigid but the posterior segment is flexible and also fitted with drawstrings to finely adjust its diameter. Although this complex ring can be used in the same circumstances as a Duran Ring, it mitigates but does not overcome the handling difficulties associated with flexible rings. Another hybrid is disclosed in U.S. Pat. No. 5,716,397, entitled “Annuloplasty Device with Removable Stiffening Element,” issued Feb. 10, 1998, to Medtronic, Inc. as the assignee of the inventor Myers. This hybrid is a flexible ring into which a rigid structure is inserted to provide temporary rigidity during implantation. Once the ring is implanted and tested, the rigid structure may be removed. However, this approach requires undesirable additional handling after the ring is implanted. Another hybrid is disclosed in U.S. Pat. No. 5,104,407, entitled “Selectively Flexible Annuloplasty Ring,” issued Apr. 14, 1992, to Baxter International, Inc. as the assignee of the inventors Lain et al., and comprises a ring constructed partially of a flexible material and partially of a rigid material. Unfortunately, this ring will be difficult and costly to manufacture and will suffer from both the drawbacks afflicting flexible rings and the those afflicting rigid rings.

The present invention is directed to resolving one or all of the problems mentioned above.

### SUMMARY OF THE INVENTION

The invention, in a first aspect, is an annuloplasty ring comprising a relatively rigid hydrogel insert and a sheath enclosing the hydrogel insert, the sheath being constructed of a biomedical material. A desired degree of initial rigidity of the annuloplasty ring is provided by maintaining a sufficiently low water content in the hydrogel insert material prior to implantation. Typically, the water content present in the hydrogel insert will be less than about 20% by weight, preferably less than about 10% by weight and more preferably less than about 5% by weight, prior to implantation. Following implantation of the annuloplasty ring into a patient, the flexibility of the hydrogel insert increases over time as the insert absorbs water from blood and/or other physiological fluids with which it comes into contact.

In a second aspect, the invention is a method of manufacturing an annuloplasty ring. The method comprises providing a relatively rigid hydrogel insert having a water content sufficiently low for providing a desired degree of initial rigidity and enclosing the hydrogel insert in a sheath constructed from a biomedical material.

### BRIEF DESCRIPTION OF THE DRAWINGS

The invention may be understood by reference to the following description taken in conjunction with the accompanying drawings, in which like reference numerals identify like elements, and in which:

FIG. 1A is an isometric view of a first embodiment of the present invention in which the annuloplasty ring is a complete ring;

FIG. 1B is an isometric view of a second embodiment of the present invention in which the annuloplasty ring is an incomplete ring;

FIGS. 2A-2B illustrate a first embodiment for the hydrogel insert of either of the annuloplasty ring embodiments in FIGS. 1A-1B;

FIGS. 3A-3B illustrate a second embodiment for the hydrogel insert of either the embodiments in FIGS. 1A-1B alternative to that in FIGS. 2A-2B; and

FIGS. 4A-4B illustrate a third embodiment for the hydrogel insert of either the embodiments in FIGS. 1A-1B alternative to those in FIGS. 2A-2B and FIGS. 3A-3B.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF THE INVENTION

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

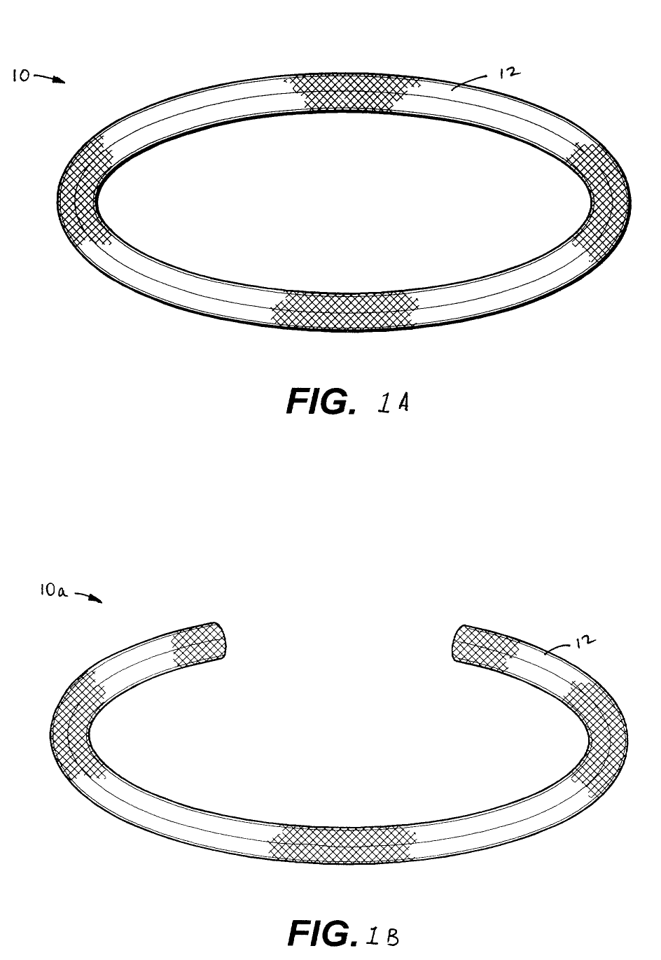

FIGS. 1A and 1B illustrate two particular embodiments 10, 10a, respectively, of the present invention. The embodiments 10, 10a are each an annuloplasty ring comprising a hydrogel insert (not shown in FIGS. 1A-1B) and a sheath 12 enclosing the hydrogel insert, the sheath 12 being constructed of a biocompatible material. The principal difference between the embodiments 10, 10a is that the embodiment 10 is a complete ring whereas the embodiment 10a is an incomplete ring. Both the embodiments 10, 10a are substantially annular in shape and are shaped in a geometry selected from the group comprising an oval and a C. More particularly, the embodiment 10 is oval-shaped and the embodiment 10a is C-shaped. However, other geometries may be employed in alternative embodiments.

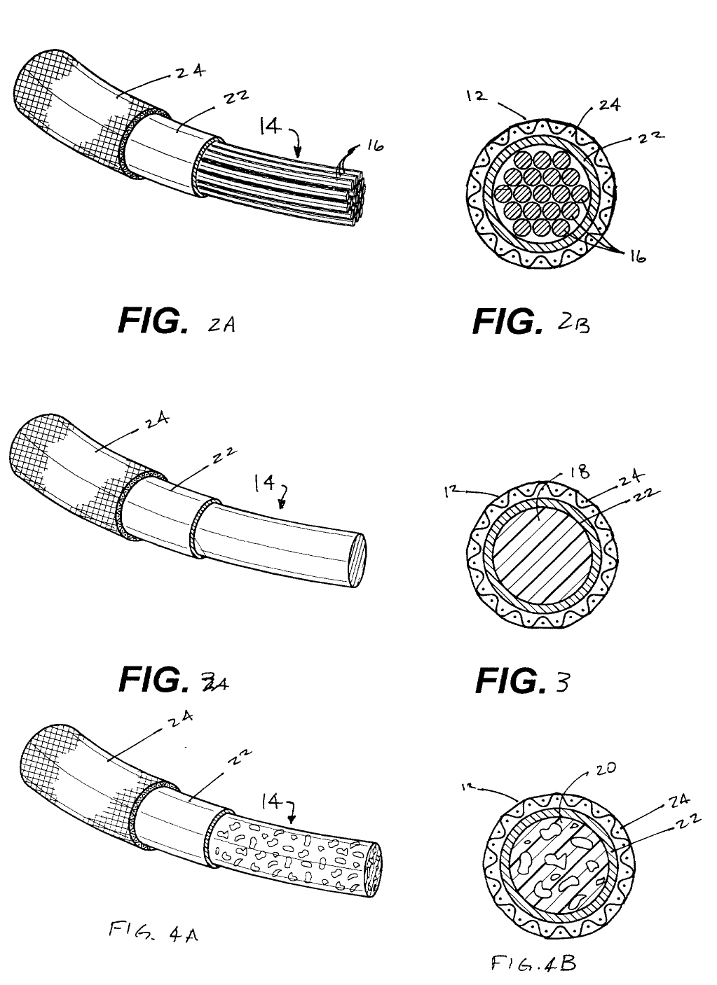

FIGS. 2A-2B, 3A-3B, and 4A-4B illustrate three different, alternative embodiments for the hydrogel insert 14. Each of the three structures may be employed as the hydrogel insert for either of the embodiments 10, 10a in FIGS. 1A-1B. For example, the hydrogel insert 14 may comprise a cabled group of fibers 16, shown in FIGS. 2A-2B, a solid rod 18, shown in FIGS. 3A-3B, or a porous rod 20, shown in FIGS. 4A-4B. However, the invention is not limited to these structures, and other suitable structures will be apparent to those skilled in the art having the benefit of this disclosure. For instance, although not shown, the hydrogel insert 14 could also be a tube in some alternative embodiments. The selection of one structure over another will be implementation and/or composition specific. Generally, the flexibility of structures such as the cabled fibers 16 and the porous rod 20 will increase more quickly following implantation of the annuloplasty ring than will the solid rod 18 since they present a proportionately higher surface area for the absorption of water molecules.

The hydrogel insert 14 may be inserted directly into the sheath 12 without any intermediary structure such that the sheath 12 comprises a single piece. However, the particular embodiments illustrated in FIGS. 2A-2B, 3A-3B, and 4A-4B each include a sheath 12 comprising a covering 22 and a fabric 24 wrapping the covering 22. The covering 22 and the fabric 24 may be constructed from the same biocompatible material or, in alternative embodiments, different biocompatible materials. As earlier mentioned, the sheath 12 may be constructed of any biocompatible material. For instance, the sheath 12, including either or both the covering 22 and fabric 24, may comprise a biomedical material selected from the group comprising silicon rubber, poly(ether urethane), polytetrafluoroethylene, polyethylene, polyethylene terephthalate, and other like materials. However, the invention is not so limited, and other suitable materials will be apparent to those skilled in the art having the benefit of this disclosure.

Hydrogels represent a known class of hydrophilic polymeric materials capable of absorbing substantial quantities of water within their structures. In general, a hydrogel material suitable for use in this invention is a material capable of absorbing water up to about 50% or more of its dry weight without substantially dissolving, and which exhibits some increase in flexibility as its water content is increased. Depending on the objectives for a given implementation and the specific hydrogel material or materials used, the hydrogel insert may be capable of absorbing large quantities of water relative to its dry weight, e.g., greater than 75%, 100%, 150%, etc., of its dry weight.

Prior to implantation, the rigidity of the hydrogel insert, and therefore the rigidity of the annuloplasty ring, is largely determined by the water content within the hydrogel insert. Thus, the initial rigidity of the annuloplasty ring can be controlled during manufacturing to meet the needs and/or preferences of the user by controlling the water content in the hydrogel insert, i.e., the lower the water content present in the hydrogel insert, the greater its rigidity. Although essentially any water content may be suitable provided it is sufficiently low to provide the desired degree of initial rigidity of the hydrogel insert, the hydrogel insert, prior to implantation, will typically contain less than about 20%, preferably less than about 10%, and more preferably less than 5% water content by weight.

The material used for fabricating a hydrogel insert according to the present invention will typically be comprised of one or more polymeric materials selected from any of a variety of hydrogel polymers, or other appropriate hydrophobic materials, known in the art. Of course, other suitable materials, e.g., interpenetrating networks, thermosets, and the like, will be readily apparent to those skilled in the art having the benefit of this disclosure, and the invention is not limited to the specific materials set forth herein.

Illustrative polymers suitable for use in the production of a hydrogel insert according to this invention may include poly-2-hydroxyethylmethacrylate (“p-HEMA”), poly-2-hydroxyethylmethacrylate/poly-N-vinyl-pyrrolidone copolymer (“p-HEMA/pNVP”), polyvinylalcohol (“PVA”), and other similar materials. These polymeric materials are readily available, are biostable, and their safety and efficacy in a number of biomedical settings is well established.

An annuloplasty ring 10, 10a may be constructed in accordance with the present invention by providing a hydrogel insert 14 as described above and enclosing the hydrogel insert 14 in a sheath 12 constructed from a biomedical material. Where the sheath 12 comprises a covering 22 and a fabric 24, enclosing the hydrogel insert 14 in the sheath 12 may include encasing the hydrogel insert 14 in the covering 22 and wrapping the covering 22 in the fabric 24.

The method of making the hydrogel insert is not specifically restricted, and is limited only by the the techniques available in the art for forming shaped articles from polymeric materials. For example, the hydrogel insert 14 may be machined, extruded, injection molded, compression molded, etc., into the desired substantially annular shape having an oval or C-shaped geometry. As would be apparent to the skilled individual in this art, the particular fabrication process employed may vary depending on the implementation-specific structure of the hydrogel insert 14, e.g., cabled fibers, porous rod, solid rod or tube, and on the manner in which the hydrogel material is synthesized, polymerized, cross-linked, etc.

The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed:

1. An annuloplasty ring, comprising:
a hydrogel insert; and
a sheath enclosing the hydrogel insert, the sheath being constructed of a biomedical material.

2. The annuloplasty ring of claim 1, wherein the hydrogel insert has a water content less than about 20% by weight.

3. The annuloplasty ring of claim 1, wherein the hydrogel insert has a water content less than about 10% by weight.

4. The annuloplasty ring of claim 1, wherein the hydrogel insert has a water content less than about 5% by weight.

5. The annuloplasty ring of claim 1, wherein the hydrogel insert is comprised of a material selected from the group comprising a polymer, an interpenetrating network, and a thermoset.

6. The annuloplasty ring of claim 1, wherein the hydrogel insert is comprised of a material capable of absorbing water at greater than about 50% of its dry weight.

7. The annuloplasty ring of claim 1, wherein the hydrogel insert is comprised of a material capable of absorbing water at greater than about 75% of its dry weight.

8. The annuloplasty ring of claim 1, wherein the hydrogel insert is comprised of a material capable of absorbing water at greater than about 100% of its dry weight.

9. The annuloplasty ring of claim 1, wherein the hydrogel insert is comprised of a material selected from the group comprising p-HEMA, p-HEMA/pNVP, and PVA.

10. The annuloplasty ring of claim 1, wherein the hydrogel insert is a complete ring.

11. The annuloplasty ring of claim 1, wherein the hydrogel insert is an incomplete ring.

12. The annuloplasty ring of claim 1, wherein the hydrogel insert is substantially annular in shape.

13. The annuloplasty ring of claim 12, wherein the hydrogel insert is shaped in a geometry selected from the group comprising an oval and a C.

14. The annuloplasty ring of claim 1, wherein the hydrogel insert comprises a structure selected from the group comprising a tube, a group of fibers, a porous rod, and a solid rod.

15. The annuloplasty ring of claim 1, wherein the sheath further comprises:
a covering enclosing the hydrogel insert; and
a fabric wrapping the covering.

16. The annuloplasty ring of claim 15, wherein the covering and the fabric wrap are constructed from different biomedical materials.

17. The annuloplasty ring of claim 16, wherein at least one of the covering and the fabric is constructed from a biomedical material selected from the group comprising silicon rubber, poly(ether urethane), polytetrafluoroethylene, polyethylene, and polyethylene terephthalate.

18. The annuloplasty ring of claim 1, wherein the sheath comprises a single piece.

19. The annuloplasty ring of claim 18, wherein the biomedical material is selected from the group comprising silicon rubber, poly(ether urethane), polytetrafluoroethylene, polyethylene, and polyethylene terephthalate.

20. The annuloplasty ring of claim 1, wherein the biomedical material is selected from the group comprising silicon rubber, poly(ether urethane), polytetrafluoroethylene, polyethylene, and polyethylene terephthalate.

21. The annuloplasty ring of claim 1, wherein the sheath is an incomplete ring.

22. The annuloplasty ring of claim 1, wherein the sheath is a complete ring.

23. A method of manufacturing an annuloplasty ring, comprising:
providing a hydrogel insert; and
enclosing the hydrogel insert in a sheath constructed from a biomedical material.

24. The annuloplasty ring of claim 23, wherein the hydrogel insert has a water content less than about 20% by weight.

25. The annuloplasty ring of claim 23, wherein the hydrogel insert has a water content less than about 10% by weight.

26. The annuloplasty ring of claim 23, wherein the hydrogel insert has a water content less than about 5% by weight.

27. The method of claim 23, wherein providing the hydrogel insert includes fabricating the hydrogel insert from a material selected from the group comprising a polymer, an interpenetrating network, and a therrmoset.

28. The method of claim 23, wherein the hydrogel insert is comprised of a material capable of absorbing water at greater than about 50% of its dry weight.

29. The method of claim 23, wherein the hydrogel insert is comprised of a material capable of absorbing water at greater than about 75% of its dry weight.

30. The method of claim 23, wherein the hydrogel insert is comprised of a material capable of absorbing water at greater than about 100% of its dry weight.

31. The method of claim 23, wherein the hydrogel insert comprises a polymer selected from the group comprising p-HEMA, p-HEMA/pNVP, and PVA.

32. The method of claim 23, wherein providing the hydrogel insert comprises molding a hydrogel material in a substantially annular shape.

33. The method of 23, wherein providing the hydrogel insert includes one of extruding a hydrogel material into a substantially annular shape, injection molding a hydrogel material into a substantially annular shape, and compression molding a hydrogel material into a substantially annular shape.

34. The method of claim 23, wherein providing the hydrogel insert includes fabricating a complete ring.

35. The method of claim 23, wherein providing the hydrogel insert includes fabricating an incomplete ring.

36. The method of claim 23, wherein providing the hydrogel insert includes fabricating a ring substantially annular in shape.

37. The method of claim 36, wherein providing the hydrogel insert includes fabricating a ring shaped in a geometry selected from the group comprising an oval and a C.

38. The method of claim 23, wherein providing the hydrogel insert includes fabricating a structure selected from the group comprising a tube, a group of fibers, a porous rod, and a solid rod.

39. The method of 23, wherein providing the hydrogel insert includes extruding a hydrogel material into a substantially annular shape.

40. The method of claim 23, wherein enclosing the hydrogel insert in the sheath includes:
encasing the hydrogel insert in a covering; and
wrapping the covering in a fabric.

41. The method of claim 40, wherein the covering and the fabric are constructed from different biomedical materials.

42. The method of claim 41, wherein at least one of the covering and the fabric is constructed from a biomedical material elected from the group comprising silicon rubber, poly(ether urethane), polytetrafluoroethylene, polyethylene, and polyethylene terephthalate.

43. The method of claim 23, wherein enclosing the hydrogel insert in the sheath includes enclosing the hydrogel insert in a sheath comprising a single piece.

44. The method of claim 43, wherein the biomedical material is selected from the group comprising silicon rubber, poly(ether urethane), polytetrafluoroethylene, polyethylene, and polyethylene terephthalate.

45. The method of claim 23, wherein the biomedical material is selected from the group comprising silicon rubber, poly(ether urethane), polytetrafluoroethylene, polyethylene, and polyethylene terephthalate.

46. The method of claim 23, wherein the sheath is a complete ring.

47. The method of claim 23, wherein the sheath is an incomplete ring.
